(19) United States
(12) Patent Application Publication
(10) Pub. No.: US 2012/0302830 A1
(43) Pub. Date: Nov. 29, 2012

(54) GUIDE ASSEMBLY FOR ENDOSCOPE

(76) Inventors: Tsuyoshi Ashida, Kanagawa (JP); Takayuki Nakamura, Kanagawa (JP); Shinichi Yamakawa, Kanagawa (JP); Yasunori Ohta, Kanagawa (JP)

(21) Appl. No.: 13/479,598

(22) Filed: May 24, 2012

(30) Foreign Application Priority Data
May 25, 2011 (JP) 2011-117066

(51) Int. Cl. A61B 1/00

(52) U.S. Cl. 600/114

(57) ABSTRACT

An endoscope includes a head assembly having a first central axis for entry in a body cavity. In combination with the endoscope, a guide assembly includes a shaft sleeve having a lumen, for mounting on the head assembly therewith. An endless track device is supported around the shaft sleeve, for endlessly moving along the first central axis, for propulsion of the head assembly inside the body cavity. A support sleeve is disposed between the shaft sleeve and the endless track device, having a second central axis different from the first central axis, for supporting the endless track device movably. Preferably, the endoscope includes an imaging window area formed in a distal surface of the head assembly and offset from the first central axis. A position shift between the first and second central axes is predetermined according to a position shift between the imaging window area and the first central axis.

### BACKGROUND OF THE INVENTION

1. Field of the Invention

The present invention relates to a guide assembly for an endoscope. More particularly, the present invention relates to a guide assembly for an endoscope, in which an imaging window of the endoscope can be positioned suitably for imaging even when the guide assembly is mounted for propulsion.

2. Description Related to the Prior Art

An endoscope is widely used for medical diagnosis. An elongated tube or guide tube of the endoscope is entered in a body cavity of a patient. A CCD image sensor or other imaging device is incorporated in the elongated tube. An image of an object in the body cavity is created by the endoscope, and is display on a display panel. A doctor or operator observes the image of the object.

U.S. Pat. Pub. No. 2005/0272976 (corresponding to JP-A 2005-253892) discloses a guide assembly for propulsion of the endoscope in the body cavity. The guide assembly includes a sleeve and a plurality of endless belts or endless track device. The sleeve is mounted on the elongated tube of the endoscope. The endless belts are supported on the sleeve movably in an axial direction. An upper run (active run) of the endless belts is kept in contact with tissue of a gastrointestinal tract, and is moved endlessly. Thus, a tip of the endoscope is propelled according to friction between the endless belts and the tissue. It is possible for a doctor or operator with insufficient skill of manipulation to enter the endoscope of the gastrointestinal tract into the body cavity, even a sigmoid colon in a large intestine or the like having a tortuous form.

The endoscope has a head assembly at its distal end. In U.S. Pat. Pub. No. 2005/0272976, the guide assembly is set radially concentric with the head assembly of the endoscope in relation to their central axes. An imaging window is formed in the head assembly of the endoscope for imaging of the body cavity. In widely used types of the endoscope, the imaging window is positioned off-center at the tip of the head assembly. The imaging window is likely to become off-center with the tip of the guide assembly for the reason of the concentricity between the guide assembly and the head assembly of the endoscope. There is a problem of difficulty in exact imaging of the body cavity due to the off-center position of the imaging window.

### SUMMARY OF THE INVENTION

In view of the foregoing problems, an object of the present invention is to provide a guide assembly for an endoscope, in which an imaging window of the endoscope can be positioned suitably for imaging even when the guide assembly is mounted for propulsion.

In order to achieve the above and other objects and advantages of this invention, a guide assembly for an endoscope having an elongated tube for entry in a body cavity is provided. There is a shaft sleeve having a lumen defined about a first central axis, for mounting on a head assembly of the elongated tube by reception thereof. A support sleeve is disposed around the shaft sleeve, defined about a second central axis different from the first central axis, for extending non-concentrically with the lumen. An endless track device is supported around the support sleeve, for endlessly moving along inner and outer surfaces of the support sleeve, for propulsion of the elongated tube relative to the body cavity.

The endoscope includes an imaging window area and a distal instrument opening disposed in a distal surface of the head assembly. A position shift between the first and second central axes is substantially equal to a position shift between a center of the distal surface and the imaging window area or between the center of the distal surface and the distal instrument opening.

In a preferred embodiment, a central axis of the shaft sleeve is the first central axis, and the support sleeve is non-concentric with the shaft sleeve.

In one preferred embodiment, the endoscope includes an imaging window area formed in a distal surface of the head assembly and offset from the first central axis. A position shift between the first and second central axes is predetermined according to a position shift between the imaging window area and the first central axis.

Furthermore, a spacer is disposed between the support sleeve and the shaft sleeve on a side in a predetermined radial direction, for keeping a wider inner space between the support sleeve and the shaft sleeve than in radial directions different from the predetermined radial direction. The predetermined radial direction is a direction of the position shift between the first and second central axes.

The shaft sleeve aligns the imaging window area with the second central axis by mounting on the head assembly.

Furthermore, a driving device drives the endless track device, and for partially constituting the spacer.

The endless track device has an annular surface, and extends to cover the support sleeve.

In one preferred embodiment, the endless track device includes plural endless belts arranged in a circumferential direction of the support sleeve.

Furthermore, a positioning device positions the head assembly in the shaft sleeve in a circumferential direction.

The head assembly includes a keyway groove. The positioning device includes at least one key projection, formed on an inner surface of the shaft sleeve, and entered in the keyway groove.

The driving device includes plural support rollers, secured to the support sleeve in a rotatable manner, for supporting the endless track device movably in contact with an inner surface thereof.

The driving device includes a drive sleeve disposed inside the support sleeve, and supported around the shaft sleeve in a rotatable manner. Worm gear teeth are formed on an outer surface of the drive sleeve. A plurality of drive gears are meshed with the worm gear teeth, for tensioning the endless track device with the plural support rollers, and driving the endless track device.

Furthermore, a barrel sleeve is supported around the shaft sleeve, surrounded by the endless track device, for supporting the plural drive gears in a rotatable manner. The plural drive gears are first to Nth drive gears arranged in a circumferential direction of the barrel sleeve, and the first drive gear has a larger diameter than the second to Nth drive gears, and constitutes the spacer.

In another preferred embodiment, the plural support rollers are first to Nth support rollers arranged in a circumferential direction of the support sleeve, and the first support roller has a larger diameter than the second to Nth support rollers, and constitutes the spacer.

In still another preferred embodiment, the shaft sleeve includes an outer surface defined about the second central axis. A lumen is defined about the first central axis, for receiving the head assembly.

Sizes of the drive gears or the support rollers are predetermined according to an inner space between the drive sleeve and the support sleeve.

A contact portion of the endless track device contacted by the drive gears and the support rollers has a higher rigidity than a remaining portion of the endless track device.

The contact portion has a larger thickness than the remaining portion.

The barrel sleeve has three walls arranged triangularly as viewed in a cross section, and at least one of the drive gears is disposed on one of the walls. The inner surface of the support sleeve is shaped triangularly as viewed in a cross section along the barrel sleeve, and the outer surface is shaped cylindrical.

Also, an endoscope system including an endoscope and a guide assembly is provided. The endoscope includes a head assembly having a first central axis for entry in a body cavity. The guide assembly includes a shaft sleeve for mounting on the head assembly. An endless track device is supported around the shaft sleeve, for endlessly moving along the first central axis, for propulsion of the head assembly inside the body cavity. A support sleeve is disposed between the shaft sleeve and the endless track device, having a second central axis different from the first central axis, for supporting the endless track device movably.

Accordingly, an imaging window of the endoscope can be positioned suitably for imaging even when the guide assembly is mounted for propulsion, because the support sleeve between the shaft sleeve and the endless track device, has the second central axis different from the first central axis of the head assembly.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a perspective view illustrating a head assembly of the endoscope and a guide assembly;

FIG. 3 is an exploded perspective view illustrating the guide assembly;

FIG. 4 is a section taken on line IV-IV of FIG. 3 illustrating the guide assembly;

FIG. 5 is a cross section illustrating the guide assembly with support rollers;

FIG. 5A is a cross section illustrating a drive gear and one support roller with a portion of an endless track device;

FIG. 6 is a cross section illustrating the guide assembly with drive gears;

FIG. 7 is a front elevation illustrating the endoscope and the guide assembly mounted thereon;

FIG. 8 is a front elevation illustrating an alternate condition of the same as FIG. 7 where an instrument opening is positioned at the center;

FIG. 9 is a cross section illustrating another preferred guide assembly in which a shaft sleeve has a non-concentrically formed lumen;

FIG. 10 is a cross section illustrating still another preferred guide assembly having one large support roller.

### DETAILED DESCRIPTION OF THE PREFERRED EMBODIMENT(S) OF THE PRESENT INVENTION

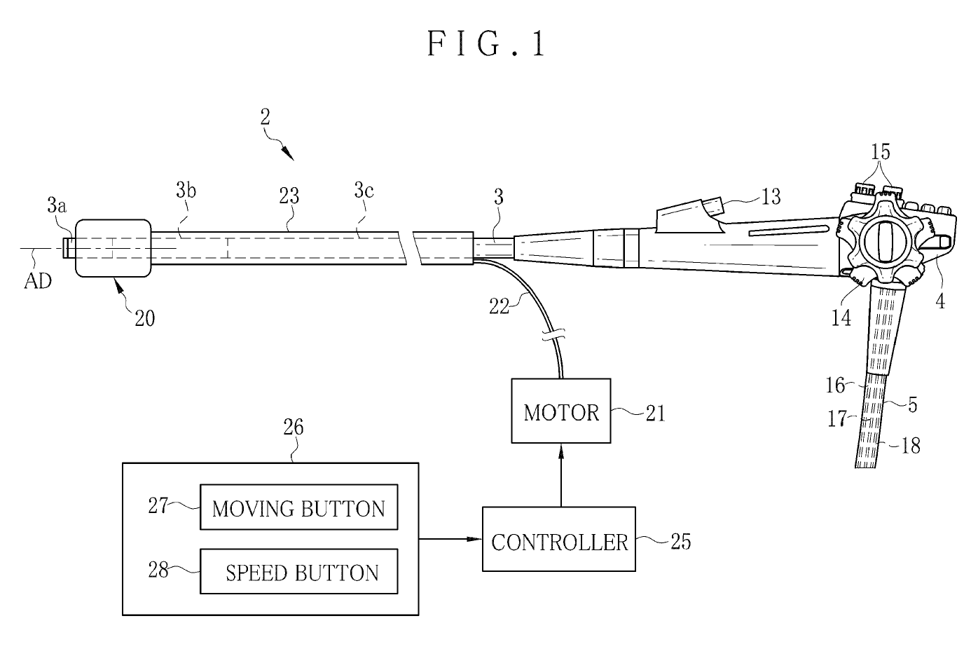

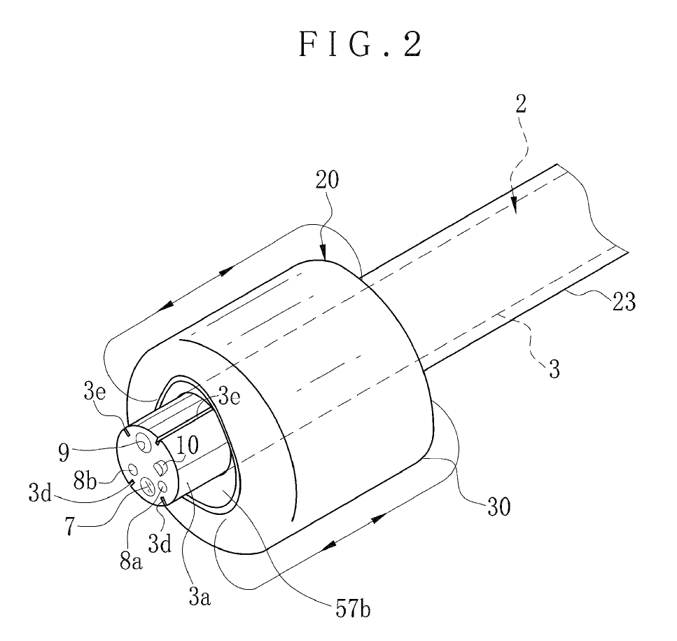

In FIGS. 1 and 2, an endoscope 2 includes a section of an elongated tube 3 or guide tube, a handle 4 and a universal cable 5. The elongated tube 3 is entered in a body cavity for imaging, for example gastrointestinal tract of a patient's body. The handle 4 is held manually by a doctor or operator and used for operating the elongated tube 3. The universal cable 5 connects the endoscope 2 to a processing apparatus, light source apparatus and fluid supply apparatus (all not shown).

The elongated tube 3 includes a head assembly 3a, a steering device 3b and a flexible device 3c. The head assembly 3a is a rigid device in which a CCD image sensor is incorporated. The steering device 3b is disposed at a proximal end of the head assembly 3a, and bendable up and down and to the right and left. The flexible device 3c extends from a proximal end of the steering device 3b with flexibility.

An imaging window area 7, lighting window areas 8a and 8b, and a distal instrument opening 9 are formed in the head assembly 3a of the elongated tube 3. The distal instrument opening 9 is used for protrusion of a tip of a forceps as a medical instrument. A nozzle spout of an end nozzle 10 is disposed on the head assembly 3a for ejection of air or water toward the imaging window area 7.

The imaging window area 7 is disposed on a distal side of the CCD image sensor for passing object light. The lighting window areas 8a and 8b are arranged symmetrically with respect to the imaging window area 7, and apply light from the light source apparatus to an object of interest in the body cavity.

An instrument channel extends from the distal instrument opening 9 in the proximal direction. A proximal instrument opening 13 is a proximal end of the instrument channel, and disposed in the handle 4. A forceps as a medical instrument is entered through the proximal instrument opening 13. Examples of tips of such medical instruments are an injection needle, electrocautery device and the like.

The handle 4 includes steering wheels 14 and control buttons 15. The steering wheels 14 are rotated to steer the steering device 3b up and down and to the right and left. The control buttons 15 are used for supplying air or water and for suction of fluid.

The universal cable 5 is connected with the handle 4. A fluid supply channel 16, a signal line 17, and a light guide device 18 are disposed through the universal cable 5. A proximal end of the fluid supply channel 16 is connected with the fluid supply apparatus. A distal end of the fluid supply channel 16 is connected to the end nozzle 10, so that the fluid supply channel 16 draws fluid from the fluid supply apparatus to the end nozzle 10. A proximal end of the signal line 17 is connected with the processing apparatus. A distal end of the signal line 17 is connected to the CCD image sensor. A distal end of the light guide device 18 is connected with the lighting window areas 8a and 8b. A proximal end of the light guide device 18 is connected to the light source apparatus, so that the light guide device 18 guides light to the lighting window areas 8a and 8b.

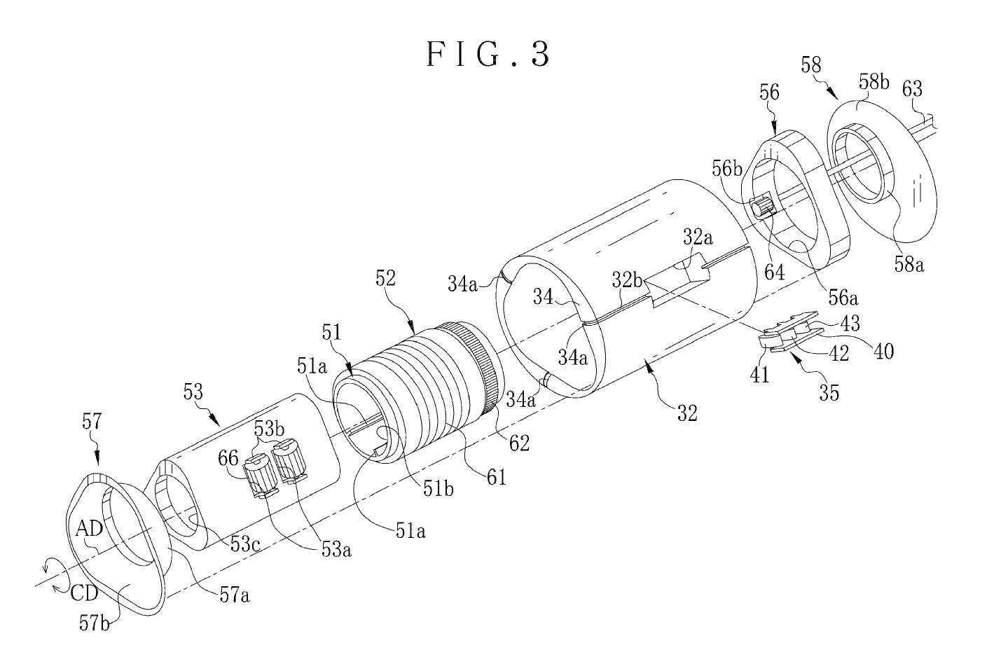

A guide assembly 20 is mounted on the head assembly 3a of the elongated tube 3 removably for propulsion (and return) of the elongated tube 3 in the gastrointestinal tract. There is a motor 21 for driving the guide assembly 20. A control wire 63 for torque of FIG. 3 is connected with an output shaft of the motor 21, and transmits torque for propulsion of the guide assembly 20. A protection sheath 22 covers the entirety of the control wire 63. When the motor 21 is actuated, the control wire 63 rotates inside the protection sheath 22.

A controller 25 controls the motor 21. A button panel 26 is connected to the controller 25, and includes a moving button 27 and a speed button 28. The moving button 27 generates a control signal for advance, return and stop of the guide assembly 20. The speed button 28 generates a control signal for changing a speed of the guide assembly 20.

An overtube 23 covers an outer surface of the elongated tube 3. The protection sheath 22 is disposed between the overtube 23 and the elongated tube 3.

In FIG. 2, an endless track device 30 is incorporated in the guide assembly 20. An upper run (active run) 36 of the endless track device 30 contacts an inner wall of a gastrointestinal tract, and moves in a proximal direction for exerting force of propulsion for the elongated tube 3 of the endoscope 2. A support sleeve 32 is an outer sleeve for supporting the endless track device 30 in a movable manner in an axial direction AD (first central axis) by turn around. The endless track device 30 extends entirely for the length of the support sleeve 32. The arrow in FIG. 2 indicates a direction of the propulsion. The endless track device 30 is formed from flexible material. Examples of the material include polyvinyl chloride, polyamide resin, fluorocarbon resin, polyurethane resin, and other biocompatible plastic compounds.

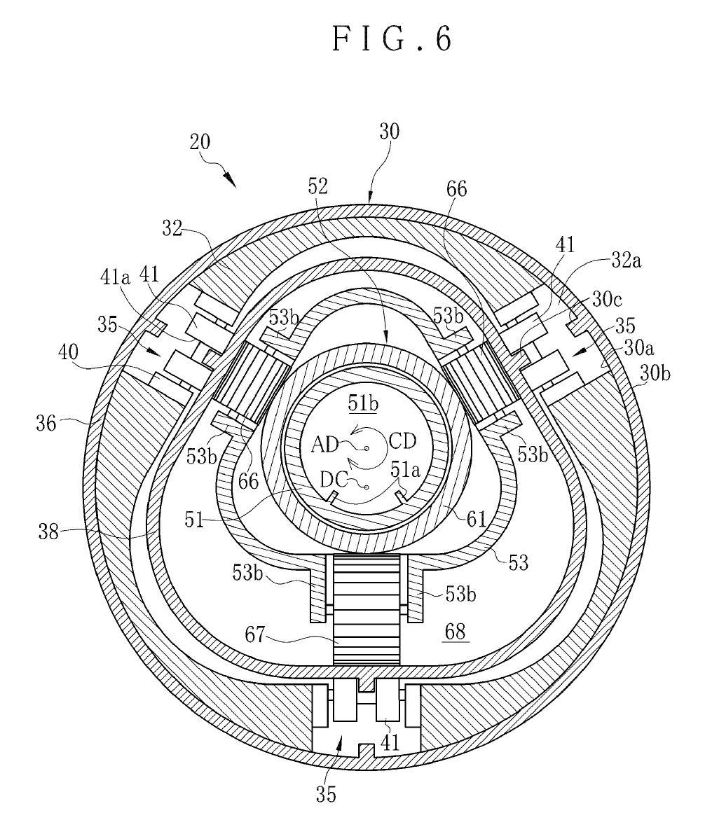

In FIGS. 3, 4, 5 and 6, an outer surface of the support sleeve 32 is circular as viewed in a cross section. An inner surface of the support sleeve 32 is triangular with curved corners as viewed in the cross section. Examples of a triangle of the triangular shape are an equilateral triangle and an isosceles triangle.

For production of the endless track device 30, an original tube material with ends is prepared. The tube material is bent inside out to follow the shape of the support sleeve 32, and is wound about the support sleeve 32. Finally, the ends are attached to one another by thermal welding or the like, to obtain the endless track device 30 in an endless form.

A curved support ring with a slip characteristic is fitted on each of proximal and distal ends of the support sleeve 32, and includes a curved support surface 34 of an annular shape, which contacts an inner surface of a bend of the endless track device 30. The curved support ring is formed from a material with the slip characteristic for keeping the endless track device 30 movable smoothly. Examples of the material include nylon, polyetheretherketone (PEEK), tetrafluoroethylene polymer or Teflon (trade name), and the like.

A side opening 32a is formed in the support sleeve 32 at each of three portions of which an inner surface is flat. A roller unit 35 is attached to the inside of the side opening 32a for contacting the endless track device 30 in a movable manner. The roller unit 35 includes two support plates 40, a first support roller 41, a second support roller 42, and a third support roller 43 or idler rollers. The support rollers 41-43 are arranged in the axial direction AD. The support plates 40 support the support rollers 41-43. It is possible to secure the support rollers 41-43 directly to the support sleeve 32 in a rotatable manner. The number of the positions of disposing the roller unit 35, although three according to the embodiment, can be determined two, or four or more for the purpose.

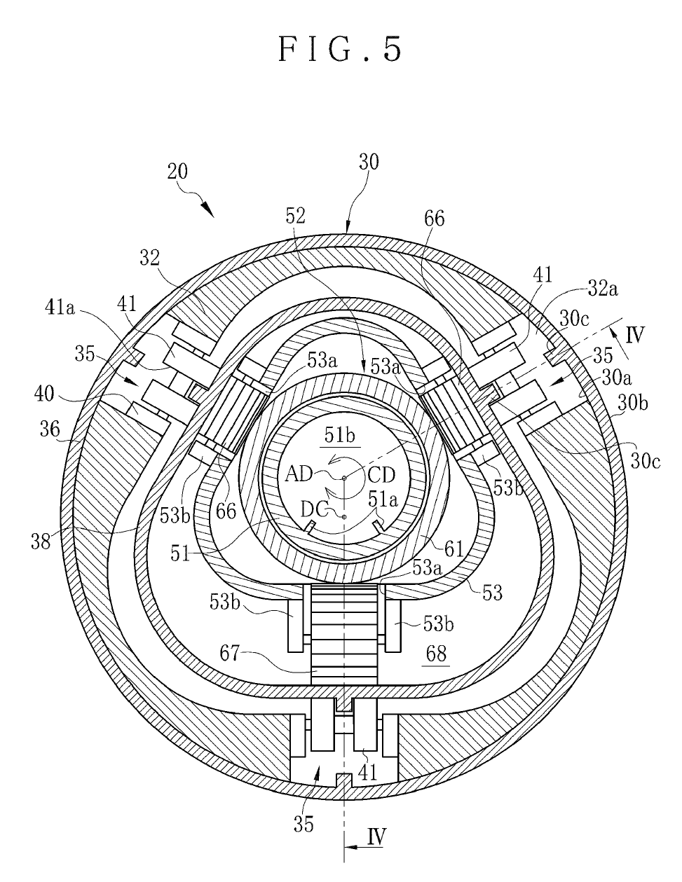

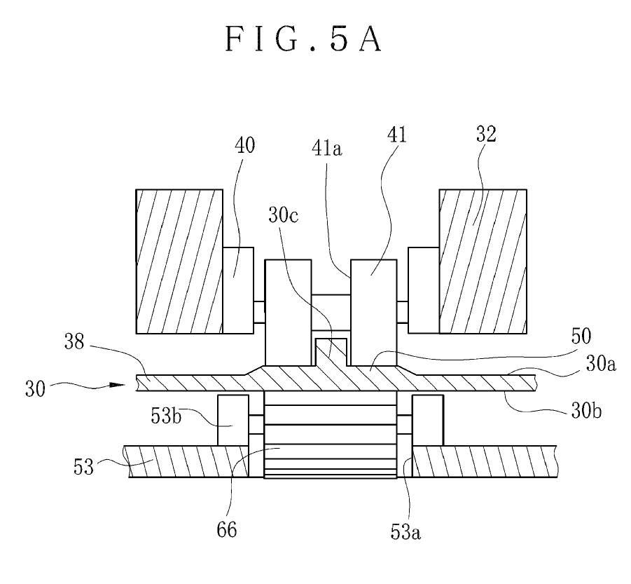

The endless track device 30 includes a lower run (return run) 38. An inner surface 30a of the lower run 38 contacts the support rollers 41-43. Three portions 50 of the endless track device 30 (See FIG. 5A) contacting the support rollers 41-43 have a larger thickness than a remaining portion of the endless track device 30, and a higher rigidity than the same.

A roller groove 41a is formed in each of the support rollers 41-43. Three engagement projections 30c or ridges are formed on the inner surface 30a of the endless track device 30. The engagement projections 30c extend fully with the length of the inner surface 30a. The roller groove 41a receives each of the engagement projections 30c in a slidable manner, and prevents the endless track device 30 from incidentally shifting in the circumferential direction CD. Also, a groove 32b is formed in the support sleeve 32. An end groove 34a is formed in the curved support surface 34. The grooves 32b and 34a receive each of the engagement projections 30c. Note that lubricant agent as a coating is applied to surfaces of the grooves 32b, 34a and 41a and the engagement projections 30c for a high slip characteristic.

Plural sleeves are disposed in the support sleeve 32, including a shaft sleeve 51, a drive sleeve 52 or threaded sleeve, and a barrel sleeve 53. The shaft sleeve 51 is mounted on the head assembly 3a of the endoscope 2 by receiving the same. The drive sleeve 52 is a driving device supported around the shaft sleeve 51 in a rotatable manner. The barrel sleeve 53 contains the shaft sleeve 51 and the drive sleeve 52 coaxially.

Two key projections 51a as a positioning device are formed inside the shaft sleeve 51 for positioning the head assembly 3a of the endoscope 2 in the circumferential direction. First keyway grooves 3d and second keyway grooves 3e are formed in an outer surface of the head assembly 3a and the steering device 3b of the endoscope 2. The first keyway grooves 3d are used for positioning the imaging window area 7 at the center of the guide assembly 20 in the radial direction. The second keyway grooves 3e are used for positioning the distal instrument opening 9 at the center. The key projections 51a are entered in one selected set of the keyway grooves 3d and 3e.

A lumen 51b is formed in the shaft sleeve 51 for receiving entry of the head assembly 3a. The shaft sleeve 51 is so disposed in the support sleeve 32 that the form of the lumen 51b is non-concentric with the form of the support sleeve 32.

An end ring 56 is secured to a proximal end of the barrel sleeve 53. A distal cover flange 57 is secured to a distal end of the barrel sleeve 53, and prevents entry of body tissue of the gastrointestinal tract. A proximal cover flange 58 is secured to a proximal end of the barrel sleeve 53 for the same purpose.

The drive sleeve 52 is supported around the shaft sleeve 51, and rotates around the axial direction AD. The drive sleeve 52 includes worm gear teeth 61 and spur gear teeth 62. The worm gear teeth 61 are a thread defined around the axial direction AD. The spur gear teeth 62 are arranged in a circumferential direction CD. The spur gear teeth 62 are formed at a proximal end of the drive sleeve 52. A pinion 64 of the control wire 63 is meshed with the spur gear teeth 62. The pinion 64 is rotated by the control wire 63, and causes the spur gear teeth 62 to rotate for the drive sleeve 52 to rotate.

The barrel sleeve 53 is in a shape of a triangular prism with curved corners. The shape of the barrel sleeve 53 is non-concentric with that of the support sleeve 32. Side openings 53a are formed in flat walls of the barrel sleeve 53. For the driving device, small drive gears 66 or engagement rollers (wheels) are disposed in two of the side openings 53a near to the inner surface of the support sleeve 32. A large drive gear 67 or engagement roller (wheel) is disposed in one of the side openings 53a distant from the inner surface of the support sleeve 32. A holder bracket 53b is formed on the barrel sleeve 53, and supports each of the drive gears 66 and 67 in a rotatable manner. Each of the drive gears 66 and 67 is disposed between the support rollers 41 and 42 or between the support rollers 42 and 43. A wide inner space 68 is defined inside the support sleeve 32 because the large drive gear 67 operates as a spacer.

An outer surface 30b of the endless track device 30 is contacted by the drive gears 66 and 67 which are in mesh with the worm gear teeth 61 of the drive sleeve 52. The endless track device 30 is tensioned between the drive gears 66 and 67 and the support rollers 41-43. Positions of the drive gears 66 and 67 are overlapped with the support rollers 41-43 in the radial direction of the support sleeve 32. The endless track device 30 is curved in a W shape between the drive gears 66 and 67 and the support rollers 41-43.

A distal opening 53c is formed at a distal end of the barrel sleeve 53, and receives a tip of the shaft sleeve 51.

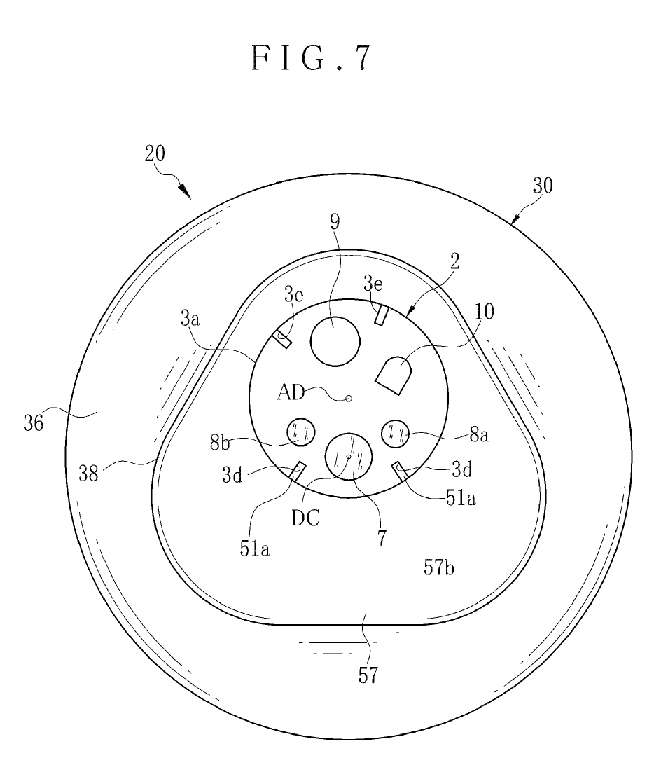

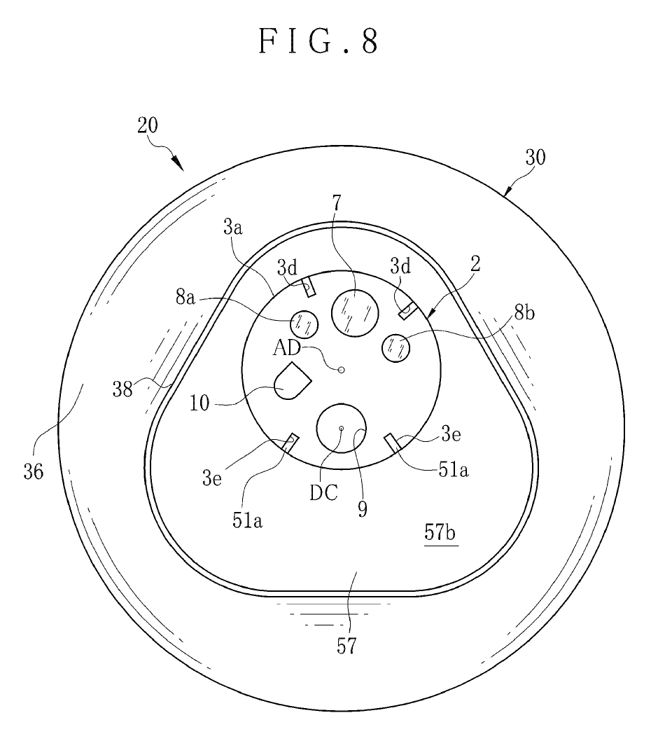

The distal cover flange 57 includes an annular ridge 57a and a cup wall 57b. The annular ridge 57a is fitted in the distal opening 53c. The cup wall 57b prevents tissue of the gastrointestinal tract from incidentally entering the guide assembly 20. The cup wall 57b is in a cup shape of which a diameter increases according to its distance from the annular ridge 57a. A shape of a section of the cup wall 57b is triangular and similar to an inner surface of the support sleeve 32, and is smaller than the support sleeve 32. The distal cover flange 57 is so formed that a center of the cup wall 57b is different from that of the annular ridge 57a. See FIGS. 7 and 8.

The end ring 56 is formed triangularly. A ring opening 56a is formed in the end ring 56 and communicates with the lumen 51b of the shaft sleeve 51. A holder recess 56b is formed in the end ring 56 and contains the pinion 64 in a rotatable manner. The pinion 64 is meshed with the spur gear teeth 62 of the drive sleeve 52. A hole (not shown) is formed through the end ring 56, and causes the control wire 63 to pass for connection to the pinion 64.

The proximal cover flange 58 is similar to the distal cover flange 57, and includes an annular ridge 58a and a cup wall 58b. The annular ridge 58a is entered in the ring opening 56a of the end ring 56. A form of the cup wall 58b is non-concentric with that of the annular ridge 58a, or has a lower center in the drawing.

The operation of the guide assembly 20 is described now. In FIG. 7, the head assembly 3a of the endoscope 2 is entered in the lumen 51b of the shaft sleeve 51 for mounting the guide assembly 20 on the head assembly 3a. To this end, the key projections 51a are entered in the first keyway grooves 3d of the head assembly 3a for setting the imaging window area 7 at the center of the guide assembly 20. Then the power source for the processing apparatus, light source apparatus and the button panel 26 is turned on for standby. The head assembly 3a of the endoscope 2 is entered in the gastrointestinal tract of the patient when the examination is ready.

When the head assembly 3a advances to a predetermined site in the gastrointestinal tract, for example, short of a sigmoid colon, the moving button 27 of the button panel 26 is depressed to input a signal for advance. Thus, the motor 21 is actuated to rotate the control wire 63 in a predetermined direction. The pinion 64 is rotated by the control wire 63, and causes the spur gear teeth 62 of the drive sleeve 52 to rotate.

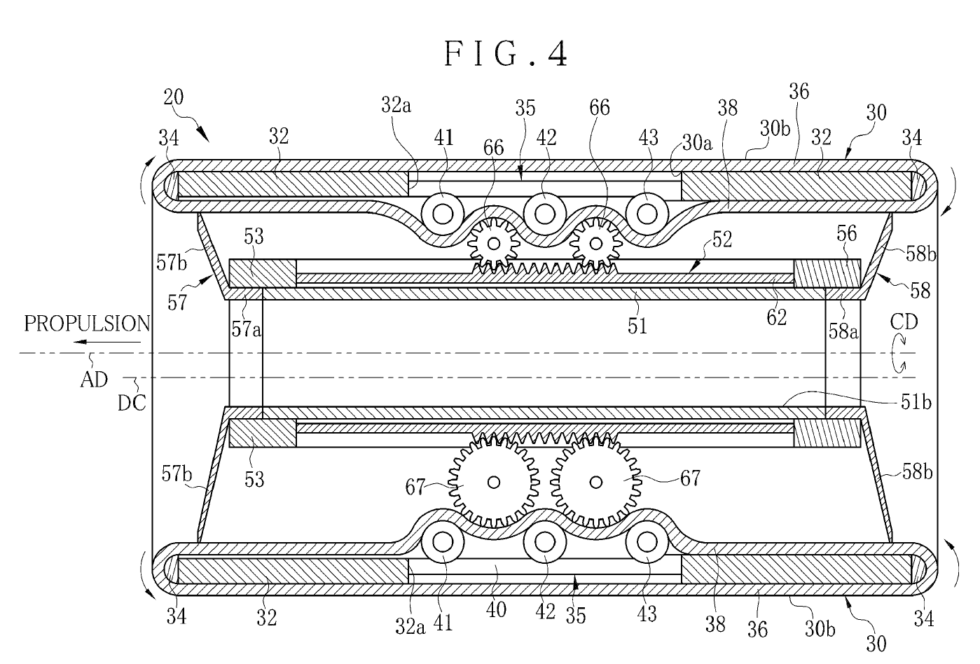

As the drive sleeve 52 is rotated, the drive gears 66 and 67 rotate in mesh with the worm gear teeth 61. In response, the lower run 38 of the endless track device 30 is moved in the propulsion direction in FIG. 4 between the drive gears 66 and 67 and the support rollers 41-43. The outer surface 30b of the upper run 36 of the endless track device 30 moves in the proximal direction outside the support sleeve 32 in contact with the body tissue of the gastrointestinal tract. The outer surface 30b of the lower run 38 inside the support sleeve 32 moves in the distal direction for turning around the endless track device 30 endlessly.

The upper run 36 of the endless track device 30 contacts body tissue of a gastrointestinal tract. When the endless track device 30 turns around, the endless track device 30 exerts force of propulsion in the proximal direction which is opposite to the distal direction of movement of the head assembly 3a. The guide assembly 20 propels the head assembly 3a of the endoscope 2 by exerting force to the body tissue from the distal side in the proximal direction. Also, the guide assembly 20 returns the head assembly 3a of the endoscope 2 by exerting force to the body tissue from the proximal side in the distal direction.

When the speed button 28 of the button panel 26 is depressed to input a control signal, the speed of the motor 21 is changed to change the moving speed of the guide assembly 20 by means of the control wire 63. When the moving button 27 of the button panel 26 is depressed to input a control signal for return, the motor 21 is rotated backwards to move the guide assembly 20 in the proximal direction by rotation of the control wire 63. When the moving button 27 is depressed to input a control signal for stop, the motor 21 is stopped to stop the guide assembly 20 by means of the control wire 63. Consequently, it is possible to propel the head assembly 3a of the endoscope 2 to a desired site in the gastrointestinal tract by combination of those functions.

Light from the light source apparatus is guided through the light guide device 18 and the lighting window areas 8a and 8b, and applied to an object in a body cavity. The CCD in the head assembly 3a detects object light from the object, and outputs an image signal. The image signal is input to a processing apparatus through the signal line 17 and the universal cable 5, for a display panel (not shown) to display an image. A doctor or operator observes the inside of the body cavity with the display panel.

When he or she discovers a lesion in the object of interest by imaging, he or she protrudes a suitable type of a medical instrument from the distal instrument opening 9 by passage through the proximal instrument opening 13 for treatment of the lesion.

To clean up the imaging window area 7, the control buttons 15 are operated to supply fluid through the fluid supply channel 16 to the end nozzle 10, for example air or water from the fluid supply apparatus. The air or water is ejected by the end nozzle 10 to the imaging window area 7 to remove dust.

As described heretofore, the form of the shaft sleeve 51 is non-concentric with that of the support sleeve 32. The imaging window area 7 is aligned with the central axis DC of the guide assembly 20 in FIG. 7. Accordingly, an object of interest can be imaged through the imaging window area 7 with greater ease than a conventional structure of the imaging window area 7 off-center with the central axis DC of the guide assembly 20.

In FIG. 8, the key projections 51a are positioned in the second keyway grooves 3e of the head assembly 3a to keep the imaging window area 7 off-center with the central axis DC of the guide assembly 20, and align the distal instrument opening 9 with the central axis DC. It is possible readily to detect an image of an object of interest from the off-center position away from the central axis DC, and to protrude a forceps or medical instrument through the central axis DC.

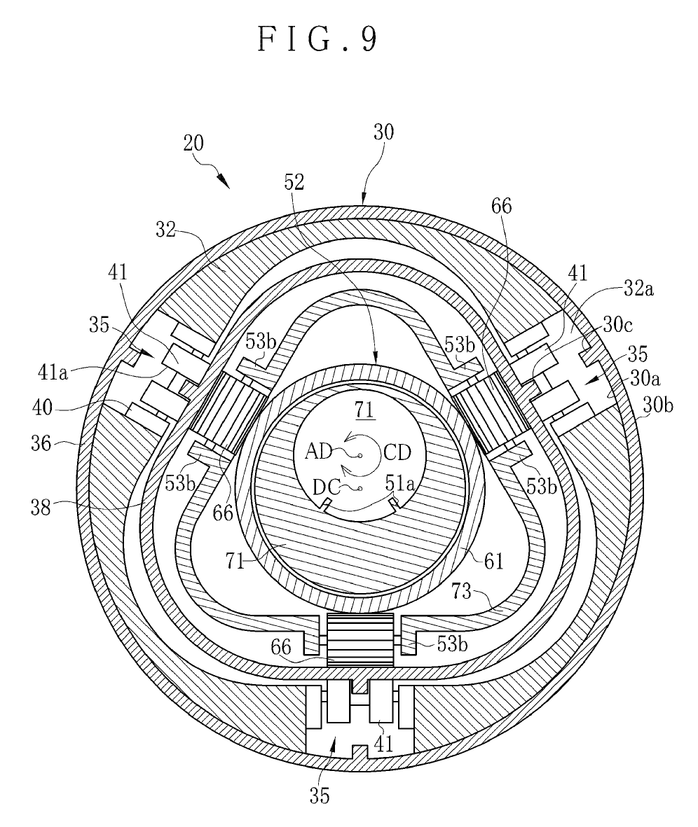

In FIG. 9, another preferred embodiment includes a shaft sleeve 71 and a barrel sleeve 73. The shaft sleeve 71 with the barrel sleeve 73 is disposed concentrically with the support sleeve 32. A lumen 71a is formed in the shaft sleeve 71 non-concentrically with an outer surface of the shaft sleeve 71.

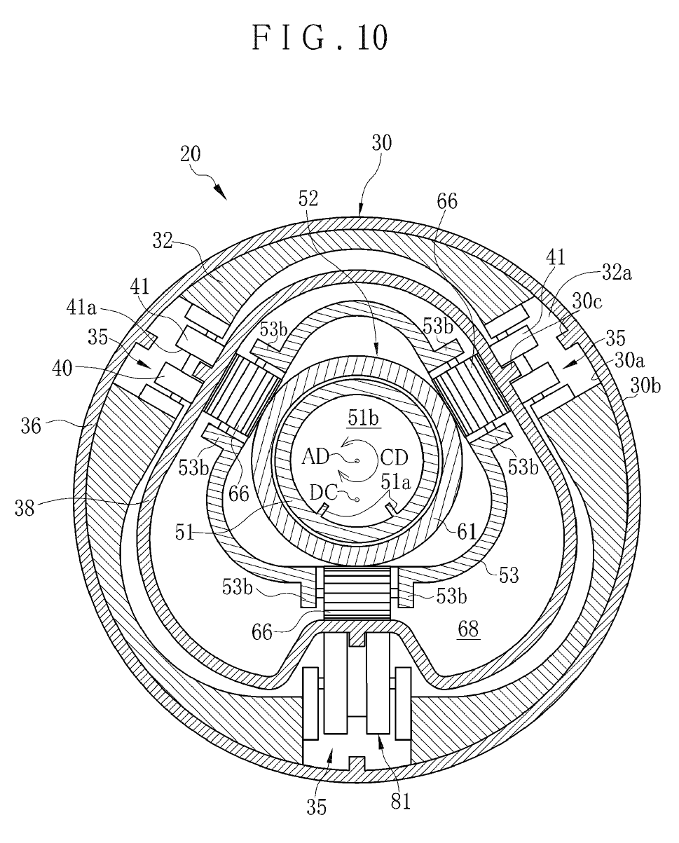

In FIG. 10, another preferred embodiment is illustrated. The small drive gears 66 are three for driving without use of the large drive gear 67 of the above embodiment. A support roller 81 or idler roller of one position has a large diameter, for use in combination with the support rollers 41-43 of two positions with the small diameter, and operates as a spacer for the wide inner space 68. In a manner similar to the above embodiment, the form of the shaft sleeve 51 is non-concentric with that of the support sleeve 32. Note that the number of the support rollers 81 in the guide assembly 20 is three arranged in the direction perpendicular to the drawing sheet of FIG. 10.

In the above embodiments, a profile surface of the support sleeve 32 is circular as viewed in a cross section. However, the support sleeve 32 can be formed in a polygonal shape, for example, triangular or quadrangular shape as viewed in a cross section.

In the above embodiments, the endless track device is in a toroidal shape. However, an endless track device of the invention may include a plurality of endless belts arranged in a circumferential direction CD of the support sleeve and extending in the axial direction.

Although the key projections 51a and the keyway grooves 3d and 3e are used for positioning the head assembly in the circumferential direction in the above embodiments, it is possible to modify a projection and groove for positioning. For example, a projection may be formed with a proximal or distal cover flange. A keyway groove may be formed in an inner surface of the lumen in the shaft sleeve 51. A positioning projection can be formed on the head assembly 3a for reception in the keyway groove. Also, the head assembly 3a can be kept rotatable in the shaft sleeve 51. A positioning ring can be added to position the head assembly 3a rotationally in a specific position as desired.

In the above embodiments, the head assembly is positioned in a selected one of the two orientations for the imaging window and the instrument opening. However, orientations for positioning the head assembly can be changed for the purpose, and can be three, four or more. Also, a mechanism having a motor, gear and the like can be used for rotating the head assembly of the endoscope to adjust the rotational position automatically.

In the above embodiments, the drive gears 66 and 67 are used between the worm gear teeth 61 and the endless track device 30. However, it is possible structurally to cause the worm gear teeth 61 to drive the endless track device 30 directly without use of the drive gears 66 and 67. To this end, circumferential directions of the motor and control wire should be determined suitably, because a circumferential direction of the worm gear teeth 61 for propulsion is opposite between structures with and without the drive gears 66 and 67.

In the above embodiments, the imaging window area 7 is aligned exactly with the central axis DC. However, it is possible to position the imaging window area 7 sufficiently near to the central axis DC by determining a difference between the central axes AD and DC.

In the above embodiments, the spacer as a feature of the invention is constituted by a part of the driving device having the drive sleeve 52 and the drive gears 66 and 67. However, other structures can be used as a spacer to define the sufficiently wide inner space 68.

In the above embodiments, the guide assembly 20 is positioned by engagement of the key projections 51a with the first keyway grooves 3d. However, the positioning device of such a structure can be omitted. It is possible for a doctor or operator manually to position the guide assembly 20 around the head assembly 3a. Also, marks can be formed instead of shaping the first keyway grooves 3d and the key projections 51a, so that he or she can align the marks with one another by visual recognition and manual handling.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly for an endoscope having an elongated tube for entry in a body cavity, comprising:
a shaft sleeve, having a lumen defined about a first central axis, for mounting on a head assembly of said elongated tube by reception thereof;
a support sleeve, disposed around said shaft sleeve, defined about a second central axis different from said first central axis, for extending non-concentrically with said lumen;
an endless track device, supported around said support sleeve, for endlessly moving along inner and outer surfaces of said support sleeve, for propulsion of said elongated tube relative to said body cavity.

2. A guide assembly as defined in claim 1, wherein said endoscope includes an imaging window area and a distal instrument opening disposed in a distal surface of said head assembly;
a position shift between said first and second central axes is substantially equal to a position shift between a center of said distal surface and said imaging window area or between said center of said distal surface and said distal instrument opening.

3. A guide assembly as defined in claim 2, wherein a central axis of said shaft sleeve is said first central axis, and said support sleeve is non-concentric with said shaft sleeve.

4. A guide assembly as defined in claim 2, wherein said first central axis is different from a central axis of said shaft sleeve so that said lumen is non-concentric with said shaft sleeve.

5. A guide assembly as defined in claim 2, further comprising a driving device for driving said endless track device;
said endless track device including:
a drive sleeve, disposed in said support sleeve, supported around said shaft sleeve, and caused to rotate by a drive source;
worm gear teeth formed around said drive sleeve;
a barrel sleeve disposed between said drive sleeve and said support sleeve;
plural drive gears, arranged on an outer surface of said barrel sleeve in a circumferential direction, rotated by said worm gear teeth, for endlessly moving said endless track device;
plural support rollers, supported on said support sleeve in a rotatable manner, for tensioning said endless track device in cooperation with said drive gears.

6. A guide assembly as defined in claim 5, wherein sizes of said drive gears or said support rollers are predetermined according to an inner space between said drive sleeve and said support sleeve.

7. A guide assembly as defined in claim 6, wherein a contact portion of said endless track device contacted by said drive gears and said support rollers has a higher rigidity than a remaining portion of said endless track device.

8. A guide assembly as defined in claim 7, wherein said contact portion has a larger thickness than said remaining portion.

9. A guide assembly as defined in claim 6, wherein said barrel sleeve has three walls arranged triangularly as viewed in a cross section, and at least one of said drive gears is disposed on one of said walls;
said inner surface of said support sleeve is shaped triangularly as viewed in a cross section along said barrel sleeve, and said outer surface is cylindrical.

10. A guide assembly as defined in claim 2, wherein said endless track device has an annular surface, and extends to cover said support sleeve along said inner and outer surfaces.

11. A guide assembly as defined in claim 2, wherein said endless track device includes plural endless belts.

12. A guide assembly as defined in claim 2, further comprising a positioning device for positioning said head assembly relative to a circumferential direction of said shaft sleeve.

13. A guide assembly as defined in claim 12, wherein said positioning device includes a key projection formed on an inner surface of said shaft sleeve, and a groove is formed in said head assembly and receives said key projection.
